(19) United States
(12) Patent Application Publication
(10) Pub. No.: US 2006/0099283 A1
(43) Pub. Date: May 11, 2006

(54) Leaf juice of plectranthus amboinicus for treating cancer and/or tumor

(75) Inventors: Cheng-Yu Wei, Hualien City (TW); Kun-Shan Lu, Jhonghe City (TW); Ching-Wu Hsia, Taipei City (TW); Huei Long, Hualien City (TW)

Correspondence Address: Ladas & Parry LLP, 26 West 61st Street, New York, NY, 10023, US

(73) Assignees: WE GENE TECHNOLOGIES, INC.; CHING-WU HSIA

(21) Appl. No.: 10/985,423

(22) Filed: Nov. 10, 2004

(51) Int. Cl. A61K 36/18; A61K 31/337

(52) U.S. Cl. 424/774; 514/449

(57) ABSTRACT

The present invention relates to a composition for treating tumor comprising an effective amount of leaf juice of Plectranthus amboinicus. The present invention also provides a method for producing the composition.

### BACKGROUND OF THE INVENTION

1. Field of the invention

The invention relates to a botanic composition for treating cancer and/or tumor. 2. Description of the Related Art

Plectranthus amboinicus, also known as Plectranthus amboinicus (Lour.) Spreng., Coleus amboinicus Lour., Coleus aromaticus Benth., Coleus crassifolius Benth., Plectranthus aromaticus (Benth.) Roxb., Coleus suganda Blanco, Coleus carnosus Hassk., and Majana amboinica (Lour.) Kuntze, belongs to the genus Labiatea and has the following common names: country borage, Cuban oregano, Indian borage, Amboini coleus, French thyme, Mexican mint, Spanish thyme, Tao-shou-hsiang, and soup mint. Plectranthus amboinicus is cultivated as a medicinal plant, potherb and condiment in the tropics (parts of Africa, India, SE Asia, West Indies, Mexico, recently southern USA). The aromatic leaves are used for flavouring meat, seasoning meat, soups, fish, local beer, being consumed as a vegetable, and being employed for washing clothes and hair. Plectranthus amboinicus is also grown for its essential oil in the Far East and as an ornamental plant.

Plectranthus amboinicus is also used in traditional remedy. For example, an infusion of the leaves (sweetened with honey) can soothe coughs and colds. In addition, the vapor obtained by rubbing the leaves vigorously helps clear blocked nose. In Taiwan, Plectranthus amboinicus is widely used for topical applications, such as burning, bites, tinea and edema, and internal applications, such as carminative and antiasthma. Furthermore, Plectranthus amboinicus is well known for its anti-microbial and anti-fungi characters for many decades. Recently, diterpene lactone genous, tritperpene genus and oleanolic acid derived from Plectranthus amboinicus were evidenced to inhibit the inflammatory response in animals through inhibiting inducible COX-1 and COX-2 activities (U.S. Patent Publication Nos. 20020068098, 20020076452, 20020077350, 20020110604, and 20030108628, and U.S. Pat. No. 6,629,835).

However, none of the prior art reference teaches or suggests that the leaf juice of Plectranthus amboinicus has effect in treating cancer and/or tumor.

### SUMMARY OF THE INVENTION

The present invention provides a composition for treating cancer and/or tumor comprising an effective amount of the leaf juice of the Plectranthus amboinicus.

The present invention also provides a method for treating cancer and/or tumor comprising administering the composition of the invention to a subject in need of such therapy.

The present invention further provides a method for producing the composition of the invention, wherein the leaf juice is produced by a method comprising the steps of:
(a) harvesting Plectranthus amboinicus leaves;
(b) washing the leaves in step (a) with distilled water;
(c) removing water from the leaves; and
(d) obtaining the leaf juice from the leaves in step (c).

One specific embodiment of the present invention is a composition for treating cancer and/or tumor comprising an effective amount of the fraction of leaf juice of Plectranthus amboinicus of a molecular weight more than 50 kD.

Accordingly the present invention also provides a method for producing the composition for treating cancer and/or tumor comprising an effective amount of the fraction of leaf juice of Plectranthus amboinicus of a molecular weight more than 50 kD, wherein the fraction of the leaf juice of plectranthus amboinicus of a molecular weight more than 50 kD is produced by a method comprising the steps of:
(a) harvesting Plectranthus amboinicus leaves;
(b) washing the leaves in step (a) with distilled water;
(c) removing water from the leaves;
(d) obtaining the leaf juice from the leaves in step (c); and;
(e) passing the leaf juice in step (d) through a filter for removing the fraction of a molecular weight less than 50 kD from the leaf juice and obtaining the fraction of a molecular weight more than 50 kD.

### BRIEF DESCRIPTION OF THE DRAWINGS

FIG. 1 illustrates the HPLC spectrogram of the leaf juice of Plectranthus amboinicus.

FIG. 2 illustrates the plot of leaf juice percentage versus HepG2 cell count as described in Example 3.

FIG. 3 illustrates the plot of day after treatment of leaf juice versus HepG2 cell count as described in Example 3.

FIG. 4 illustrates the plot of leaf juice percentage versus Huh7 cell count as described in Example 3.

FIG. 5 illustrates the plot of day after treatment of leaf juice versus Huh7 cell count as described in Example 3.

FIG. 6 illustrates the plot of day after treatment of leaf juice versus Bowes cell count as described in Example 4.

FIG. 7 illustrates the result of cell count change of HepG2 treated with leaf juice and/or paciltaxel on day 3 as described in Example 5.

FIG. 8 illustrates the result of cell count change of Huh7 treated with leaf juice and/or paciltaxel on day 3 as described in Example 5.

FIG. 9 illustrates the result of tumor area change of the Huh7 inoculated mice treated with the leaf juice as described in Example 6.

FIG. 10 illustrates the result of body weight change of the Huh7 inoculated mice treated with the leaf juice as described in Example 6.

FIG. 11 illustrates the result of tumor area change of the Huh7 inoculated mice after stopping treatment of the leaf juice as described in Example 6.

FIG. 12 illustrates the result of body weight change of the Huh7 inoculated mice after stopping treatment of the leaf juice as described in Example 6.

FIG. 13 illustrates the result of tumor area change of the Bowes inoculated mice treated with the leaf juice as described in Example 7.

FIG. 14 illustrates the result of body weight change of the Bowes inoculated mice treated with the leaf juice as described in Example 7.

FIG. 15 illustrates the result of tumor area change of the Bowes inoculated mice after stopping treatment of the leaf juice as described in Example 7.

FIG. 16 illustrates the result of body weight change of the Bowes inoculated mice after stopping treatment of the leaf juice as described in Example 7.

FIG. 17 illustrates the plot of percentages of leaf juice and fractions thereof versus HepG2 cell count as described in Example 8.

FIG. 18 illustrates the result of cell count change of HepG2 treated with leaf juice and fractions thereof as described in Example 8.

FIG. 19 illustrates the HPLC spectrogram of the fraction of the leaf juice of Plectranthus amboinicus of a molecular weight more than 50 kD.

FIG. 20 illustrates the result of cell count change of HepG2 treated with leaf juice and fractions thereof as described in Example 9.

FIG. 21 illustrates the plot of percentages of leaf juice and fractions thereof versusHepG2 cell count as described in Example 10.

FIG. 22 illustrates the result of cell count change of Huh7 treated with leaf juice and fractions thereof as described in Example 10.

FIG. 23 illustrates the result of cell count change of Huh7 treated with leaf juice and fractions thereof as described in Example 11.

### DETAILED DESCRIPTION OF THE INVENTION

According to the invention, leaf juice of Plectranthus amboinicus is surprisingly found to have dramatic effects in treating cancer and/or tumor.

According to the present invention, a composition for treating cancer and/or tumor comprising an effective amount of the leaf juice of Plectranthus amboinicus is provided.

As used herein, the term “leaf juice,” also known as “leaf extract,” refers to the fluid naturally contained in leaf. The leaf juice can be obtained by removing tissue fragments and/or residues.

As used herein, the term “tumor” refers to a morbid swelling, prominence, or growth, on any part of the body; especially, a growth produced by deposition of new tissue; a neoplasm. As used herein, the term “malignant tumor,” also known as “malignant neoplastic disease” or “cancer,” refers to a tumor growth caused by abnormal and uncontrolled cell division; it may spread to other parts of the body through the lymphatic system or the blood stream.

The term “effective amount” as used herein refers to an amount of a composition which, when administered to an animal, has a desired effect on the animal. For example, an effective amount of a composition for treating tumor is an amount that controls tumor growth and/or clears tumor in the animal.

In one embodiment of the invention, the composition according to the invention is used for inhibiting malignant tumor growth. In another embodiment of the invention, the number of malignant tumor cells such as HepG2, Huh7 and Bowes is lowered and even eliminated when treated with the composition according to the invention.

When screening tumor cell lines responding to the composition according to the invention, it is found that the composition regulates tumor growth through p53 network. The composition according to the invention inhibits cell growth of the tumor cell whose p53 gene is functional including wild-type, partial functional and mutant active. Therefore, the tumor treated according to the invention is a p53 related tumor.

The p53 network is a molecular sensor for G₁ checkpoint in the cell cycle and monitors DNA damage, nucleotide pool levels, mitotic spindle status and genotoxic stress. The p53 network also regulates cell cycle progression, programmed cell death, replicative senescense and possibly differentiation. Therefore, p53 is regarded as a tumor suppressor gene. In fact, more than half of all human cancers are associated with one or more alterations in p53. Alternatively, the mutant p53 protein may have acquired a new tumor promoting activity which is independent of wild-type p53. Several strategies for treating malignant tumor directing to p53 network have been developed (U.S. Pat. Nos. 5,382,510, 5,840,579, 6,183,964, 6,472,385, and 6,531,512).

In one embodiment of the invention, the tumor to be treated comprises hepatocellular carcinoma and melanoma. More preferably, the tumor to be treated comprises hepatocellular carcinoma and melanoma related to p53 network.

The leaf juice of Plectranthus amboinicus according to the invention can be analyzed and identified with high performance liquid chromatography (HPLC). For instance, a spectrogram of 50 μL of the leaf juice of Plectranthus amboinicus taken at 214 nm through high performance liquid chromatography using a ZORBAX™ C18 column with an inner diameter (I.D.) of 4.6 mm, a length (L) of 15 cm comprises peaks at retention time of 1.756, 2.573, 7.118, 7.851, 9.715, 10.278, 10.864, 11.212, 12.287, 12.799, 13.178, 13.413, 14.027, 14.794, 16.253, and 18.742 minutes, where the leaf juice is separated with a linear gradient elution of 0-30 % acetonitrile and 0.1 % TFA in 30 min and a flow rate is 1 mL/min.

When investigating the pharmaceutical effect of the fractions of the leaf juice of Plectranthus amboinicus, it is found that the fraction of a molecular weight more than 50 kD has a stronger effect than other fractions in treating cancer and/or tumor. In one embodiment of the invention, 0.001% of the fraction of a molecular weight more than 50 kD shows a strong ability to inhibit hepatocellular carcinoma (HepG2) growth, while other fractions require a dosage of up to 0.5 % to attain similar effects.

The fraction of a molecular weight more than 50 kD according to the invention can be analyzed and identified with high performance liquid chromatography. For instance, a spectrogram of 50 μL of the fraction of a molecular weight more than 50 kD taken at 214 nm through high performance liquid chromatography using a ZORBAX™ C18 column with an inner diameter (I.D.) of 4.6 mm, a length (L) of 15 cm comprises peaks at retention time of 2.574, 4.798, 5.728, 7.101, 9.701, 10.279, 10.808, 11.189, 12.235, 13.35, 13.584, 13.903, 14.113, 15.114, 16.206, 16.736, 18.619, 22.137, 24.676, and 26.902 minutes, where the fraction of a molecular weight more than 50 kD is separated with a linear gradient elution of 0-30 % acetonitrile and 0.1 % TFA in 30 min and a flow rate is 1 mL/min.

In an embodiment of the invention, the composition further comprises an antineoplastic agent. Since tumor growth involves complicated network co-working, combining antineoplastic agents directed to different networks can reasonably elevate therapeutic effects. In a preferred embodiment of the invention, the antineoplastic agent is paciltaxel. Paciltaxel (also known as taxol), which is a clinical anticancer drug for its strong mitotic arrest characters in late G₂M phases, exhibits a strong anti-neoplastic character through mediating Bcl-2 phosphorylation and paciltaxel-induced apoptosis mechanism. Furthermore, paciltaxel is found to increase the blockage of mitosis by binding to the microtubules (Lanni S. Jennifer, Lowe W. Scott, Licitra J. Edward, Liu O. Jun, Jacks Tyler. P53-independent aopotosis induced by paciltaxel through an indirect mechanism. Proc. Natl. Acad. Sci. USA Vol 94, September 1997:9979-9683). In one embodiment of the invention, the composition comprising 5% of the leaf juice of Plectranthus amboinicus and 50 nM paciltaxel has a stronger effect in inhibiting tumor growth than the composition comprises the leaf juice of Plectranthus amboinicus or paclitaxel alone. It implies that cross-talking of p53 and Bcl-xL apoptotic pathways is an effective way in treating cancer and/or tumor.

In order to apply conveniently, the composition according to the invention is formulated in a pharmaceutically acceptable carrier.

As used herein, a “pharmaceutically acceptable carrier” includes any and all solvents, dispersion media, isotonic and absorption delaying agents, and the like. These pharmaceutically acceptable carriers may be prepared from a wide range of materials including, but not limited to diluents, binders and adhesives, lubricants, disintegrants, coloring agents, bulking agents, and miscellaneous materials such as buffers and absorbents that may be needed in order to prepare a particular therapeutic composition. The use of such media and agents for pharmaceutically active substances is well known in the art. Except insofar as any conventional media or agent is incompatible with the active ingredients, its use in the present composition is contemplated.

The composition according to the invention can be administrated to a subject through several ways. Preferably, the composition is applied to a subject via injection, oral administration, or topical administration.

A method for producing the composition according to the invention is also provided. Particularly, the leaf juice is produced by a method comprising the steps of:
(a) harvesting Plectranthus amboinicus leaves;
(b) washing the leaves in step (a) with distilled water;
(c) removing water from the leaves, preferably by air dry; and
(d) obtaining the leaf juice from the leaves in step (c).

In one embodiment of the invention, the leaves are treated to be small pieces in conventional manners such as grinding, stirring, disturbing, cutting or mincing.

Optionally, the method for producing the leaf juice further comprises step (d2) of centrifuging the leaf juice in step (d) to remove the tissue fiber. In one embodiment of the invention, centrifugations at 15,000 rpm for 5 minutes at 24° C. are conducted twice.

Optionally, the method for producing the leaf juice further comprises a step of sterilizing the leaf juice. In one embodiment of the invention, the leaf juice is sterilized by filtration.

Optionally, the method for producing the leaf juice further comprises a step of concentrating the leaf juice.

The invention also provides a composition for treating cancer and/or tumor comprising an effective amount of the fraction of the leaf juice of Plectranthus amboinicus of a molecular weight more than 50 kD.

A method for producing the composition for treating cancer and/or tumor comprising an effective amount of the fraction of the leaf juice of Plectranthus amboinicus of a molecular weight more than 50 kD, wherein the fraction of the leaf juice of Plectranthus amboinicus of a molecular weight more than 50 kD is produced by a method comprising the steps of:
(a) harvesting Plectranthus amboinicus leaves;
(b) washing the leaves in step (a) with distilled water;
(c) removing water from the leaves;
(d) obtaining the leaf juice from the leaves in step (c); and
(e) passing the leaf juice in step (d) through a filter for removing the fraction of a molecular weight less than 50 kD from the leaf juice and obtaining the fraction of a molecular weight more than 50 kD fraction.

The method for producing the fraction of the leaf juice of Plectranthus amboinicus of a molecular weight more than 50 kD is similar to that for producing the as mentioned above except step (e). The methods for separating fractions according to molecular weight ranges are well developed in the field. One embodiment of the invention uses Amicon® Ultra centrifugal filter device where filters with different pore sizes are provided for separating fractions according to molecular weight.

The following examples are given for the purpose of illustration only and are not intended to limit the scope of the present invention.

### EXAMPLE 1

### Preparation of Leaf Juice of Plectranthus amboinicus

Plectranthus amboinicus was obtained from Taiwan Endemic Species Research Institute (NanTao, Taiwan). Plectranthus amboinicus was cultured with large quantity of water. Leaves of Plectranthus amboinicus were harvested, washed with distilled water, and air-dried to remove the water residue. The tissues were ground to fine particles with juice. The leaf juice was reduced with centrifugation at 15,000 rpm for 5 minutes at 24° C. The clear solution was collected and centrifuged at 15,000 rpm for another 5 minutes at 24° C. to remove the tissue fibers as possible. The juice was filtered with 0.22 μm syringe filters to sterilize. The fresh leaf juice was stored at 4° C. in dark before use. All the plant crude extracts were used within 3 days or kept at −80° C. for long-term storage.

The chromatographic measurement was performed on an Agilent® 1100 series liquid chromatographic system consisting of 1100 Quaternary Pump with Degasser, 1100 Variable Wavelength Detector, and 1100 Standard Autosampler. Further peak analysis was performed using ChemStation® software.

Bulk solvents and mobile phases were filtered through a 0.22 μm nylon membrane filter (Alltech® Associates, Pty. Ltd) using a Millipore® solvent filtration apparatus (Millipore®, Bedford, Mass.).

The column was equilibrated with 0.1% TFA (trifluoroacetic acid), Milli-Q at a flow rate of 1 ml/min. 50 μL of 1× crude sample in PBS was injected into a ZORBAX® C18 column (4.6 mm i.d.×15cm), and the column was kept in a column oven with temperature set at 25° C. Following injection, the analytes were separated with a linear gradient elution of 0-60% acetonitrile and 0.1% TFA in 30 min. The analytes are detected at 214 nm.

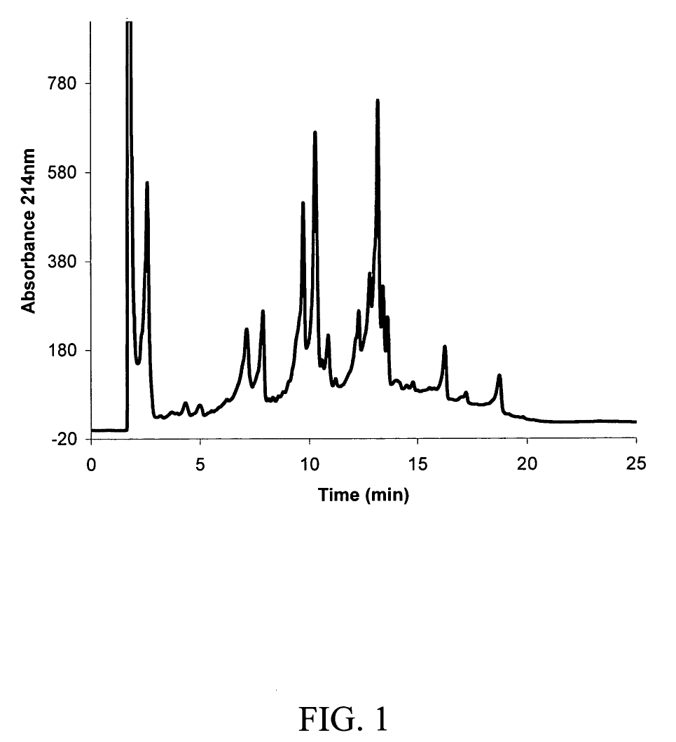

The result of chromatography is shown in FIG. 1 and the marked peaks are summarized in Table 1.

TABLE 1

| | Retention | | | |
|---|---|---|---|---|
| Peak No. | Time (min) | Area (mAU) | Area (%) | Height (mAU) |
| 1 | 1.756 | 32537.46 | 22.74 | 3233.69 |
| 2 | 2.573 | 10804.05 | 7.55 | 548.64 |
| 3 | 7.118 | 7722.92 | 5.40 | 226.42 |
| 4 | 7.851 | 6428.48 | 4.49 | 261.98 |
| 5 | 9.715 | 14589.61 | 10.20 | 505.16 |
| 6 | 10.278 | 13956.58 | 9.75 | 667.49 |
| 7 | 10.864 | 3185.86 | 2.65 | 211.76 |
| 8 | 11.212 | 1983.91 | 1.39 | 114.65 |
| 9 | 12.287 | 9357.61 | 6.54 | 167.46 |
| 10 | 12.799 | 7150.80 | 5.00 | 351.98 |
| 11 | 13.178 | 11427.11 | 7.99 | 723.62 |
| 12 | 13.413 | 6133.80 | 4.29 | 324.05 |
| 13 | 14.027 | 3307.24 | 2.31 | 111.19 |
| 14 | 14.794 | 2816.79 | 1.97 | 107.32 |
| 15 | 16.253 | 5357.76 | 3.74 | 187.43 |
| 16 | 18.742 | 5725.88 | 4.00 | 121.51 |

### EXAMPLE 2

### Screening Cell Lines Responding to the Leaf Juice

Cell lines: The following nine human tumor cell lines of different histologic origins were obtained from commercial cell culture collections unless indicated otherwise: HepG2 (haptocellular carcinoma, liver; provided by Dr. C H Huang, National Health Research Institute, Taipei, Taiwan), Hep3B (haptocellular carcinoma, liver; provided by Dr. C H Huang, National Health Research Institute, Taipei, Taiwan), U937 (myeloid leukemia), K562 (chronic myeloid leukemia, bone marrow), Calu-1 (epidermoid carcinoma, lung; provided by Dr. Y C Kuo, National Institute of Traditional Chinese Medicine, Taipei, Taiwan), TL (trophoblast-like cell line, placenta; provided by Dr. C K Ho, Veteran General Hospital, Taipei, Taiwan), Jurket (human lymphoblastoid T cell line), Bowes (human melanoma cell), and Huh7 (hepatocellular carcinoma/ectoderm).

HepG2 and Hep3B cells were cultured in minimum essential medium supplemented with 10% fetal calf serum, 2 mM L-glutamine, 50 IU/mL penicillin, and 50 μg/mL streptomycin. U937 and K562 cells were maintained in RPMI-1640 medium supplemented with 10% fetal calf serum, 2 mM L-glutamine, 50 IU/mL penicillin, and 50 μg/mL streptomycin. Calu-1 and TL cells were cultured in high-glucose Dulbecco's modified Eagle medium supplemented with 10% fetal calf serum, 2 mM L-glutamine, 50 IU/mL penicillin, and 50 μg/mL streptomycin.

Cultures were maintained at 37° C. in a humidified atmosphere containing 5% CO₂. All cell reagents were obtained from Gibco-Invitrogen Co., Ltd (Grand Island, N.Y. U.S.A.) and HyClone (Logan, Utah, U.S.A.).

Treatment: In all in vitro experiments, 3% of the leaf juice prepared according to Example 1 was added to the cells after subcultured directly.

Cell count: Total cell counts were made by adding 20 μL of the cell suspension to 20 μL of 0.4% trypan blue, and counted under a phase contrast microscope.

The result is shown in Table 2. “O” represents that the cells respond to the leaf juice (i.e. cell count decrease after treatment); “X” represents that the cells do not respond to the leaf juice (i.e. cell count increase after treatment).

TABLE 2

| | | | Response |
|---|---|---|---|
| | | | to leaf |
| Cell line | Source | p53 | juice |
| U937 | Myelomonocytic | mutant inactive | X |
| | leukemia/Mesoderm | | |
| K562 | Erythroleukemia/Mesoderm | negative | X |
| Calu-1 | Lung epidermoid carcinoma/ | deleted | X |
| | Endoderm | | |
| TL | Trophoblastoma/Endoderm | negative | X |
| Jurkat | Human lymphoblastoid T | negative | X |
| | cell line | | |
| Bowes | Human Melanoma Cell | Mutant partial | ◯ |
| | | active | |
| HepG2 | Hepatocellular | Wild type | ◯ |
| | carcinoma/Ectoderm | | |
| Huh7 | Hepatocellular | Mutant active | ◯ |
| | carcinoma/Ectoderm | | |
| Hep3B | Hepatocellular | Null | X |
| | carcinoma/Ectoderm | | |

According to the result as shown in Table 2, the cell lines whose p53 gene is functional respond to the leaf juice. It evidenced that the leaf juice regulates tumor growth through p53 network.

### EXAMPLE 3

### Leaf Juice for Treating Hepatocellular Carcinoma

The hepatocellular carcinoma cell lines used in the example are HepG2 and Huh7, and the manipulations of the cell culture and treatment are as described in Example 2. The cells were grouped into a control group (without treatment) and experiment groups treated with 1%, 3%, 5%, 7% or 9% of leaf juice. Each group was repeated eight times (HepG2) or four times (Huh7).

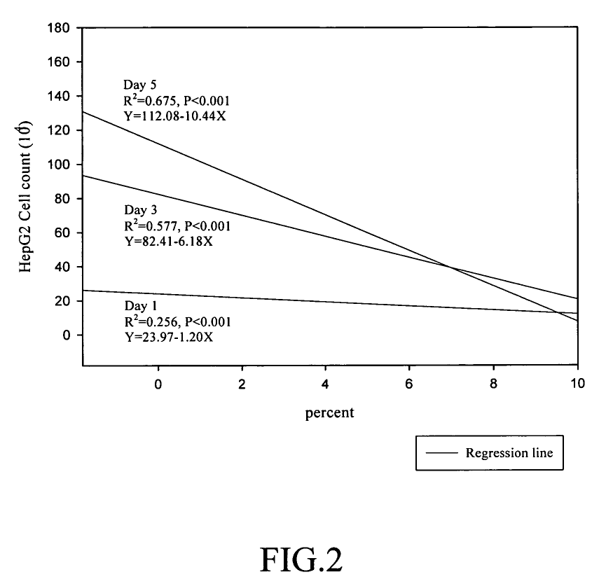

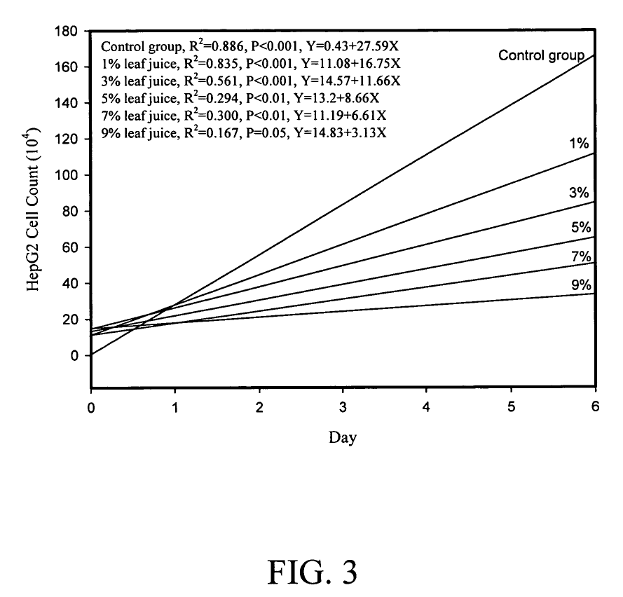

Cell counts of HepG2 cell of each group on days 1, 3 and 5 were determined and shown in FIGS. 2 and 3 and Table 3. Referring to FIGS. 2 and 3, the inhibiting effect of leaf juice increases with dose dependency on day 1 (P<0.001), day 3 (P<0.001), and day 5 (P<0.001). Additionally, the inhibiting effect of leaf juice increases as duration of treatment increases on day 1 (inhibited cell count/% of leaf juice: 1.2×10⁴), day 3 (inhibited cell count/% of leafjuice: 6.18×10⁴ ), and day 5 (inhibited cell count/% of leaf juice: 10.44×10⁴).

TABLE 3

| | Cell growth rate (10⁴ | Inhibition (compared |
|---|---|---|
| Group | cells/day) | with the control group) |
| Control group | 27.59 (P < 0.001) | — |
| 1% leaf juice | 16.75 (P < 0.001) | 0.6071 |
| 3% leaf juice | 11.66 (P < 0.001) | 0.4226 |
| 5% leaf juice | 8.66 (P < 0.01) | 0.3139 |
| 7% leaf juice | 6.61 (P < 0.01) | 0.2396 |
| 9% leaf juice | 3.13 (P = 0.05) | 0.1135 |

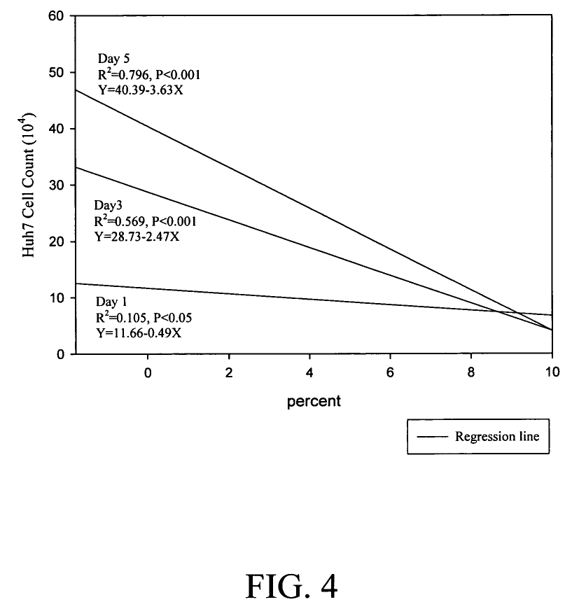

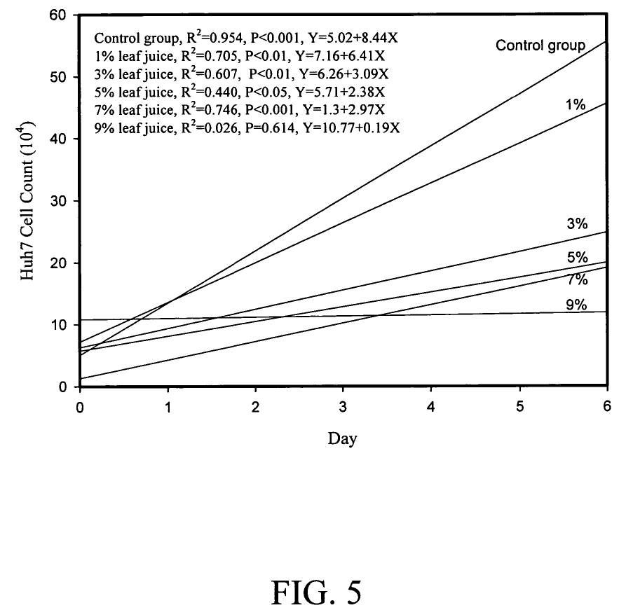

Cell counts of Huh7 cell of each group on days 1, 3 and 5 were determined and shown in FIGS. 4 and 5 and Table 4. Referring to FIGS. 4 and 5, the inhibiting effect of leaf juice increases with dose dependency on day 1 (P<0.05), day 3 (P<0.001), and day 5 (P<0.001). Additionally, the inhibiting effect of leaf juice increases as duration of treatment increases on day 1 (inhibited cell count/% of leaf juice: 0.49×10⁴), day 3 (inhibited cell count/% of leaf juice: 2.47×10⁴), and day 5 (inhibited cell count/% of leaf juice: 3.63×10⁴).

TABLE 4

| | Cell growth rate (10⁴ | Inhibition (compared |
|---|---|---|
| Group | cells/day) | with the control group) |
| Control group | 8.44 (P < 0.001) | — |
| 1% leaf juice | 6.41 (P < 0.01) | 0.7595 |
| 3% leaf juice | 3.09 (P < 0.01) | 0.3661 |
| 5% leaf juice | 2.38 (P < 0.05) | 0.2820 |
| 7% leaf juice | 2.97 (P < 0.001) | 0.3519 |
| 9% leaf juice | 0.19 (P = 0.614) | 0.0225 |

### EXAMPLE 4

### Leaf Juice for Treating Melanoma

The melanoma cell line used in the example was Bowes, and the manipulations of the cell culture and treatment were as described in Example 2. The cells were grouped into a control group (without treatment) and experiment groups treated with 1% or 3% of leaf juice. Each group was repeated five times.

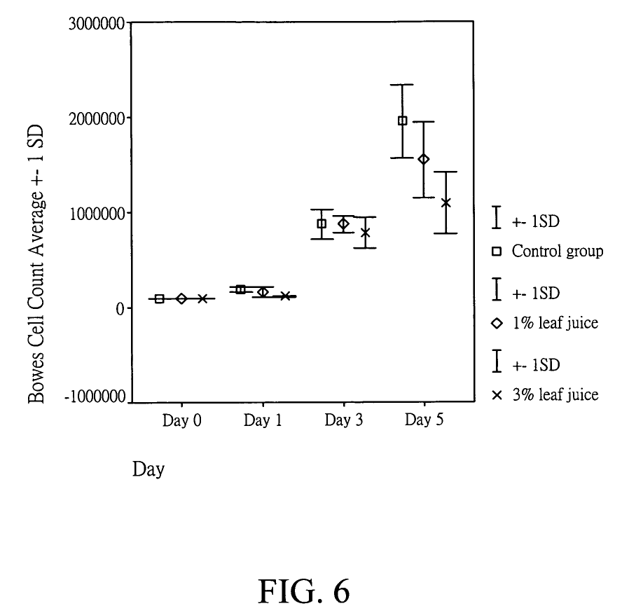

Cell counts of Bowes cell of each group on days 1, 3 and 5 were determined and shown in FIG. 6. Referring to FIG. 6, the inhibiting effect of leaf juice increases with strong dose denpendency on day 1 (P<0.05) and day 5 (P<0.005). Additionally, the inhibiting effect of leaf juice increases as duration of treatment increases on day 1 (inhibited cell count/% of leaf juice: 2.22×10⁴), day 3 (inhibited cell count/% of leaf juice: 3.26×10⁴), and day 5 (inhibited cell count/% of leafjuice: 25.8×10⁴).

### EXAMPLE 5

### Leaf Juice and/or Paciltaxel for Treating Hepatocellular Carcinoma

The hepatocellular carcinoma cell lines used in the example are HepG2 and Huh7, and the manipulations of the cell culture and treatment are as described in Example 2. The cells were grouped into a control group (without treatment) and experiment groups treated with 5% of leaf juice, 50 nM paciltaxel, and combining 5% of leaf juice and 50 nM paciltaxel. Each group was repeated six times.

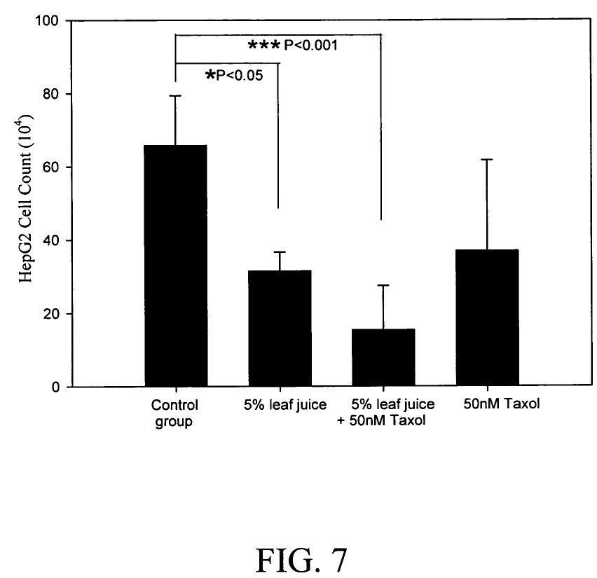

Cell count of HepG2 cell of each group on day 3 was determined and shown in FIG. 7. Referring to FIG. 7, it shows that cell growth was significantly inhibited in all experiment groups. The inhibiting rate of each experiment group is 52.13% (5% of leaf juice: P=0.017), 43.77% (50 nM paciltaxel: P=0.052), or 76.57% (combining 5% of leaf juice and 50 nM paciltaxel P=0.000). It shows that combination of the leaf juice according to the invention and paciltaxel exhibits stronger effect of cell growth inhibition than the leaf juice or paciltaxel solely.

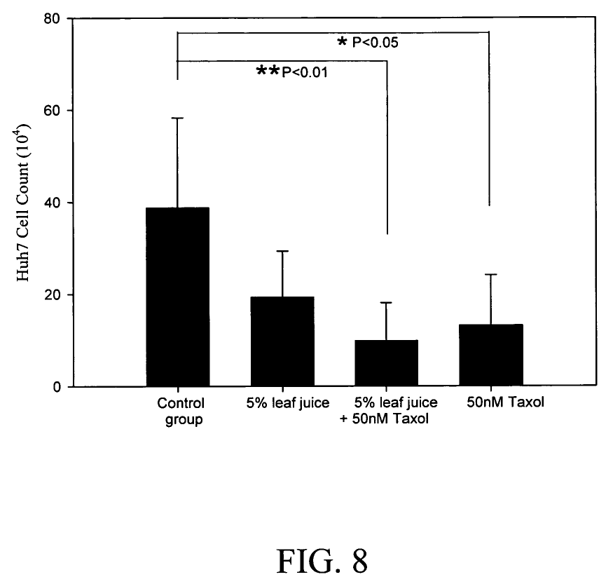

Cell count of Huh7 cell of each group on day 3 was determined and shown in FIG. 8. Referring to FIG. 8, it shows that cell growth was significantly inhibited in all experiment groups. The inhibiting rate of each experiment group is 50.21% (5% of leaf juice: P=0.110), 66.09% (50 nM paciltaxel: P=0.023), or 74.68% (combining 5% of leaf juice and 50 nM paciltaxel P=0.009). It shows that combination of the leaf juice according to the invention and paciltaxel exhibits stronger effect of cell growth inhibition than the leafjuice or paciltaxel solely.

### EXAMPLE 6

### Animal Model of Leaf Juice for Treating Hepatocellular Carcinoma

Inoculation: Huh7 cells were cultured to obtain 3×10⁸ cells in total with the condition mentioned in Example 2. The cells were then suspended in DMEM in a cell density of 2×10⁸ cells. 0.1 mL of cell solution was inoculated to the subdermal area of right back of NOD-scid mice aged 5 to 6 weeks and weighing about 20 g. In a control group, the mice were inoculated with 0.1 mL of DMEM.

Treatment: The mice with large tumor (size larger than 10 mm×10 mm, one mouse), medium tumor (size 7 mm×7 mm to 10 mm×10 mm, one mouse) or small tumor (size smaller than 7 mm×7 mm, two mice) were treated with the leaf juice prepared according to the method of Example 1. The dosage of leaf juice was 10% × mice body weight. The needle of the syringe with the leaf juice was penetrated in the subdermal space of peritumor area, and then the leaf juice was injected intra-tumor. The treatment along with tumor sizing and body weighting were performed on every Monday, Wednesday and Friday. In a blank group, 10% of PBS was administrated.

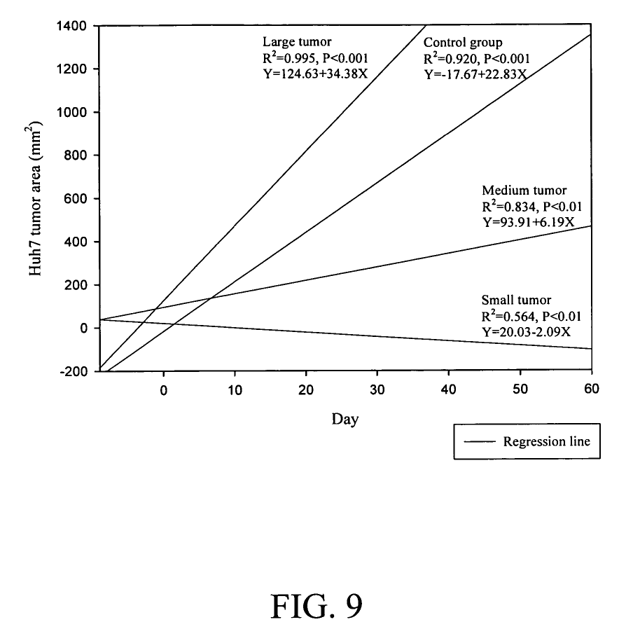

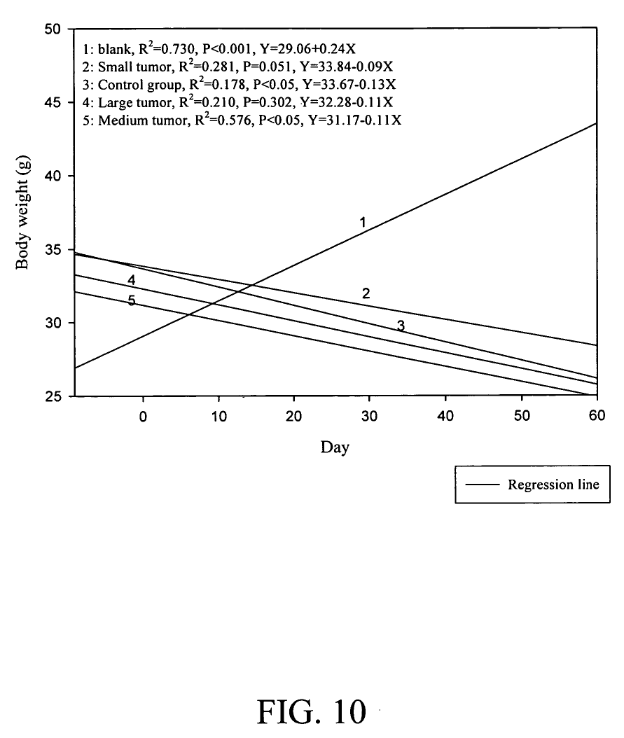

Result: The result is shown in FIG. 9. After treated with the leaf juice, the small tumor area was reduced (R²=0.564, B=−2.09 mm²/day, P<0.01). The growth of medium tumor (R²=0.834, B=6.19 mm²/day, P<0.01) was inhibited and only 27.1% of that of control group. However, the growth of large tumor (R²=0.995, B=34.38 mm²/day, P<0.01) was not inhibited. Additionally, the body weight of all of mice treated with the lead juice was reduced (without inoculation: R²=0.178, B=−0.13 g/day, P<0.05; small tumor: R²=0.281, B=−0.09 g/day, P=0.051; medium tumor: R²=0.576, B=−0.11 g/day, P<0.05; large tumor: R²=0.210, B=−0.11 g/day, p=0.302). Only the mice without treatment gained weight (R=0.730, B=0.24 g/day, P<0.001) (as shown in FIG. 10).

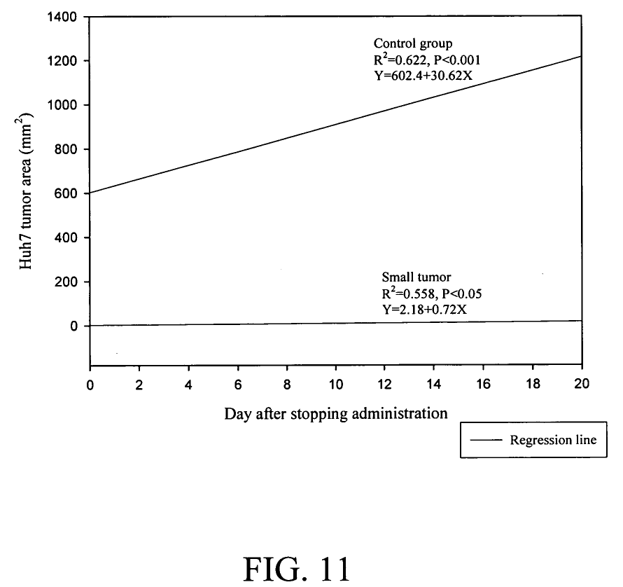

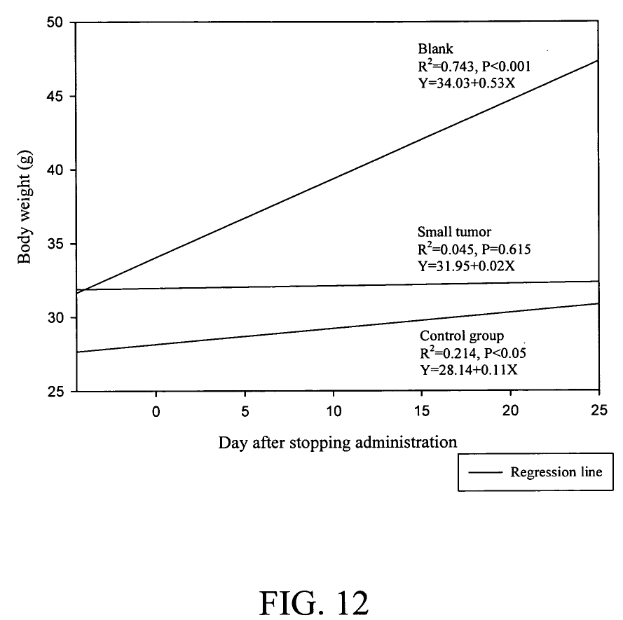

Recurrence: The administration of the group with small tumor was stopped on day 13, and the tumor area was monitored continually. The result is shown on FIG. 11. It shows that the small tumor growths (R²=0.558, B=0.72 mm²/day, P<0.05) with a growth rate of only 2.4% of that of control group (R²=0.622, B=30.62 mm²/day, P<0.01). Furthermore, all mice gained weight after administration was stopped (blank: R²=0.743, B=0.53 g/day, P<0.001; small tumor: R²=0.045, B=0.02 g/day, P<0.615 ; control group: R²=0.214, B=0.11 g/day, P<0.05) (as shown in FIG. 12).

### EXAMPLE 7

### Animal Model of Leaf Juice for Treating Melanoma

Inoculation: Bowes cells were cultured to obtain 3×10⁸ cells in total with the condition mentioned in Example 2. The cells were then suspended in DMEM in a cell density of 2×10⁸ cells. 0.1 mL of cell solution was inoculated to the subdermal area of right back of NOD-scid mice aged 5 to 6 weeks and weighted about 20 g. In a control group, the mice were inoculated with 0.1 mL of DMEM.

Treatment: The mice with large tumor (size larger than 10 mm×10 mm, one mouse) or small tumor (size smaller than 10 mm×10 mm, two mice) were treated with the leaf juice prepared according to the method of Example 1. The dosage of leaf juice was 10% × mice body weight. The needle of the syringe with the leaf juice was penetrated in the subdermal space of peritumor area, and then the leaf juice was injected intra-tumor. The treatment along with tumor sizing and body weighting were performed on every Monday, Wednesday and Friday. In a blank group, 10% of PBS was administrated.

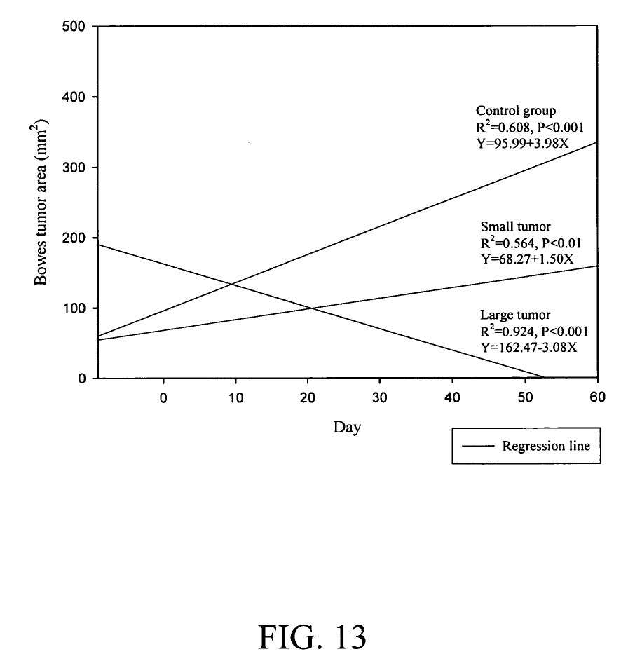

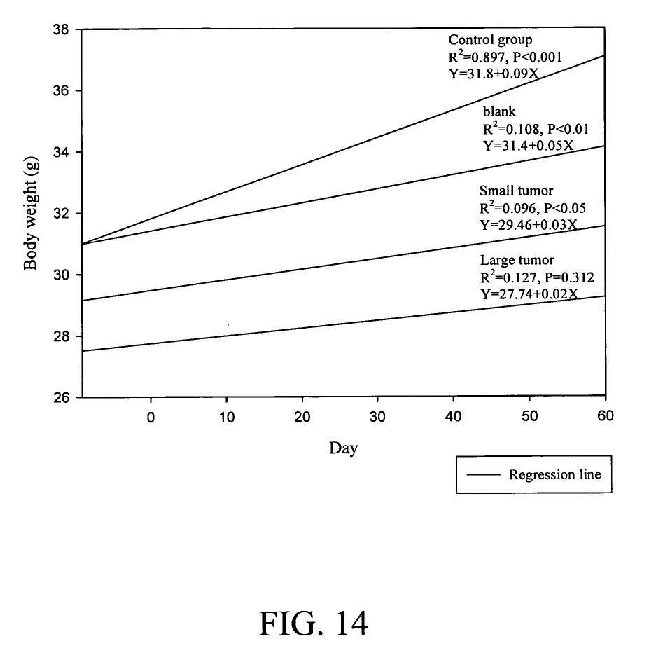

Result: The result is shown in FIG. 13. After treated with the leaf juice, the large tumor area was reduced (R²=0.924, B=-3.08 mm²/day, P<0.01). The growth of small tumor (R²=0.564, B=1.50 mm²/day, P<0.01) was inhibited and only 37.7% of that of control group. Additionally, the body weight of all of the mice treated with the lead juice was lightly increased except the mice without treatment which gained weight significantly (R²=0.897, B=0.09 g/day, P<0.001) (as shown in FIG. 14).

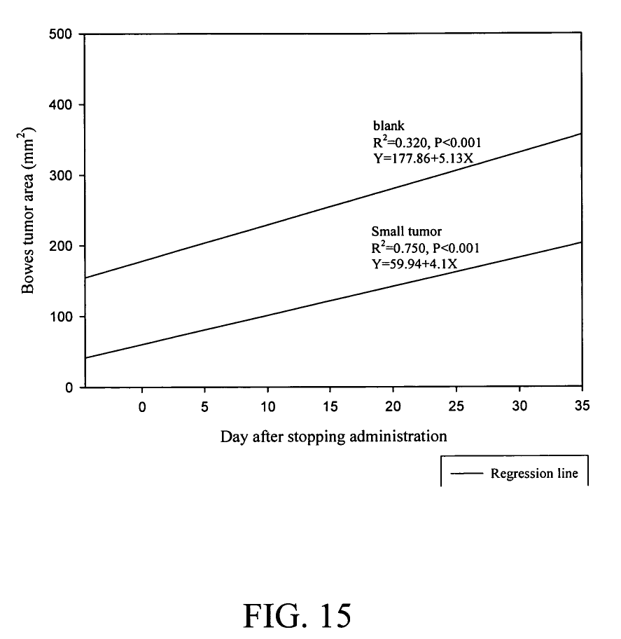

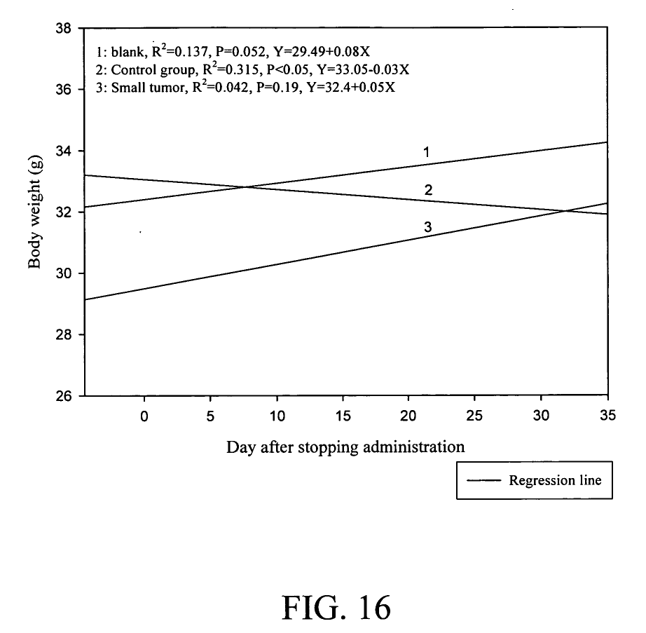

Recurrence: The administration of the group with small tumor was stopped on day 24, and the tumor area was monitored continually. The result is shown on FIG. 15. It has shown that the growth rate of small tumor after stopping administration (R²=0.750, B=4.1 mm²/day, P<0.001) was greater by 173% than that of administration (R²=0.564, B=1.50 mm²/day, P<0.01). Furthermore, the growth rate of tumor of blank mice after stopping PBS administration (R²=0.320, B=5.13 mm²/day, P<0.001) was greater by 29% than that of administration (R²=0.608, B=3.98 mm²/day, P<0.001). The small tumor group and blank group had similar growth rates after discontinuing administration. Additionally, the body weights of all the mice after stopped treating with the leaf juice lightly increased while the blank group gained the weights (R²=0.315, B=-0.03 g/day, P<0.05) more significantly than the small tumor group (as shown in FIG. 16).

### EXAMPLE 8

### Effects of Fractions of Leaf Juice in Inhibiting HepG2 Cell Growth

Fractions: The leaf juice as prepared in Example 1 was divided into Fractions #1 (molecular weight<10 K, 25× leaf juice), #2 (molecular weight<10 kD, 1× leaf juice), #3 (molecular weight 10 kD to 30 kD, 25× leaf juice), #4 (molecular weight 10 kD to 30 kD, 1× leaf juice), #5 (molecular weight 30 kD to 50 kD, 25× leaf juice), #6 (molecular weight 30 kD to 50 kD, 1× leaf juice), #7 (molecular weight <50 kD, 25× leaf juice), #8 (molecular weight>50 kD, 25× leaf juice), and #9 (molecular weight>50 kD, 1× leaf juice) with Amicon® Ultra centrifugal filter devices (Amicon Ultra PL-10, 10,000 Nominal Molecular Weight Limit (NMWL); Amicon Ultra PL-30, 30,000 NMWL; Amicon Ultra PL-50, is 50,000 NMWL, Millipore®).

Treatment: The cell line used for screening the fractions of leaf juice is HepG2. The manipulation of the cell lines is as described in Example 2 and the cell numbers were counted on day 4.

The cell viability of HepG2 on day 4 is illustrated in Table 5.

TABLE 5

| | 1% of drug | 5% of drug |
|---|---|---|
| Control | 100 | 100 |
| 1 X leaf juice | 88 | 47 |
| 25 X leaf juice | 33 | 35 |
| Fraction #1 | 11 | 14 |
| Fraction #2 | 59 | 18 |
| Fraction #3 | 74 | 27 |
| Fraction #4 | 92 | 96 |
| Fraction #5 | 57 | 57 |
| Fraction #6 | 90 | 100 |
| Fraction #7 | 98 | 33 |
| Fraction #8 | 80 | 0 |
| Fraction #9 | 13 | 0 |

It shows that Fractions #1, #2, #3, #7, #8, and #9 inhibit tumor growth with dose dependency. Furthermore, Fractions #2 and #9 have stronger growth inhibition effects on HepG2 cell line compared to other fractions.

1× and 25× leaf juice, 0%, 0.01%, 0.1%, 0.5%, 1% and 3% of Fractions #1, #2 and #9 were further used for treating HepG2 cells. Each group was repeated four to seven times.

The result shows that 0.5% (P <0.05), 1% (P<0.001) and 3% (P<0.001) Fraction #2, 0.01% Fraction #9 (P<0.001), 1% leaf juice (P<0.05) and 1% (P<0.01) and 3% (P<0.001) 25× leaf juice inhibit HepG2 growth significantly. On the other hand, 1% 1× leaf juice (P=0.848) and 1% Fraction #1 (P=0.052) do not have significant inhibiting effect. It proves that Fraction #9 has the strongest effect where the effective dosage is only 0.01%. Fraction #2 also has strong effect where the effective dosage is 0.5%. The effective dosage of 25× leaf juice is 1%. The inhibiting rates of 0.5%, 1% and 3% Fraction #2, 0.01% Fraction #9, 3% 1× leaf juice, and 1% and 3% 25× leaf juice are 34.3%, 62.8%, 98.7%, 39.7%, 54.8%, 53.6%, and 83.8%, respectively.

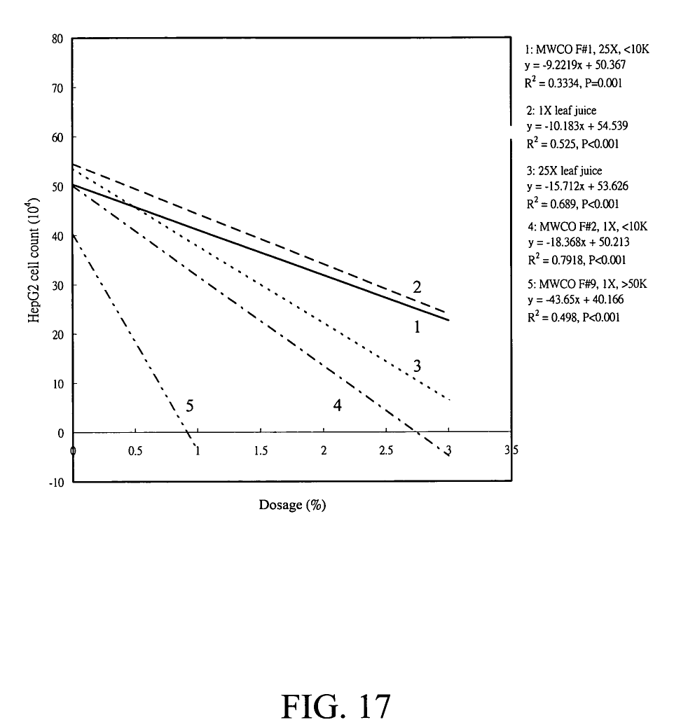

The cell numbers inhibited per percentage of 1× and 25× leaf juice and Fractions #1, #2 and #9 are shown in FIG. 17

It shows that 1× (P<0.001, R²=0.525) and 25× (P=0.001, R²=0.689) leaf juice and Fractions #1 (P<0.001, R²=0.333), #2 (P<0.001, R²=0.792) and #9 (P<0.001, R²=0.498) significantly inhibit HepG2 growth in dose dependency. The cell numbers inhibited per percentage of 1× and 25× leaf juice and Fractions #1, #2 and #9 on day 4 are 10.2, 15.7, 9.22, 18.4, and 43.7×10^{4,} respectively. The inhibiting effects are Fraction #9>Fraction #2>25× leaf juice>1× leaf juice>Fraction #1.

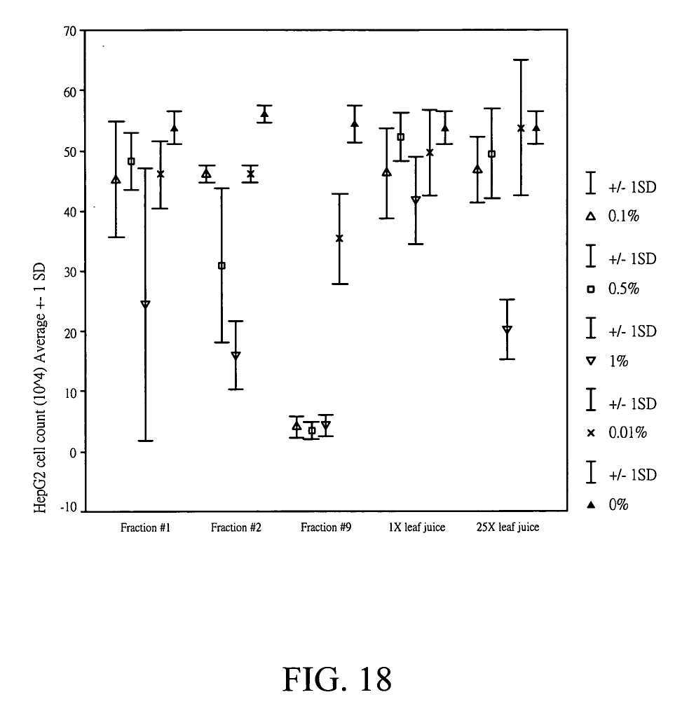

The effects of 1× and 25× leaf juice, Fractions #1, #2 and #9 with the same dosage are also analyzed. The result is shown in FIG. 18.

When the dosages are 0.1% and 0.5%, the effect of Fraction #9 is stronger than those of 1× and 25× leaf juice, and Fractions #1 and #2 significantly (all P<0.001).

When the dosage is 1%, the effect of Fraction #9 is significantly stronger than that of 1× leaf juice (P=0.001) and significantly than those of 25× leaf juice, and Fractions #1 (P=0.059) and #2 (P=0.072). Furthermore, the effect of Fraction #2 is stronger than that of 1× leaf juice significantly (P=0.05).

When the dosage is 0.001%, the effect of Fraction #9 is stronger than that of 25× leaf juice significantly (P=0.063).

When the dosage is 0.1%, the effect of Fraction #9 is stronger than those of 1× and 25× leaf juice, and Fractions #1 and #2 significantly (all P<0.001).

### EXAMPLE 9

### Fraction #9 of Leaf Juice for Treating Hepatocellular Carcinoma HepG2

The chromatographic measurement of Fraction #9 was performed as described in Example 1.

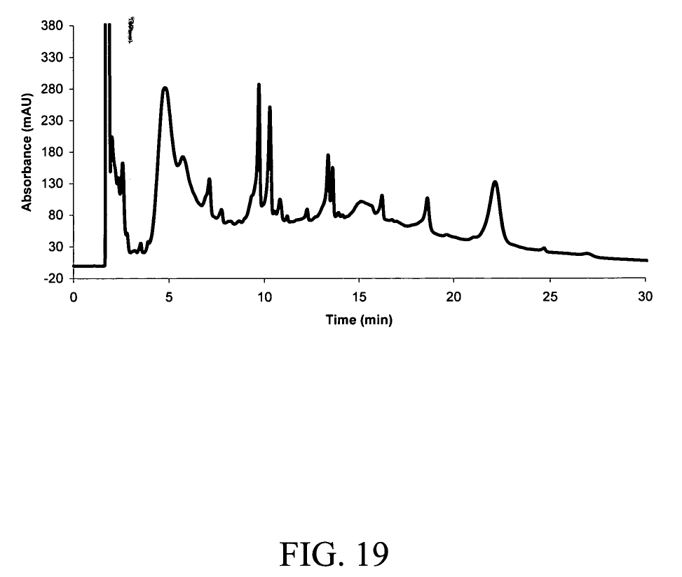

The result of chromatography is shown in FIG. 19 and the marked peaks are summarized in Table 6.

TABLE 6

| Peak No. | Retention Time (min) | Area (mAU) | Height (mAU) |
|---|---|---|---|
| 1 | 2.574 | 2729.02 | 161.45 |
| 2 | 4.798 | 17088.54 | 283042 |
| 3 | 5.728 | 10144.49 | 174.31 |
| 4 | 7.101 | 4715.61 | 137.99 |
| 5 | 9.701 | 7839.76 | 282.37 |
| 6 | 10.279 | 5654.36 | 251.75 |
| 7 | 10.808 | 2310.79 | 108.42 |
| 8 | 11.189 | 1406.56 | 81.95 |
| 9 | 12.235 | 4973.73 | 92.76 |
| 10 | 13.35 | 6341.88 | 177.22 |
| 11 | 13.584 | 2012.7 | 156.81 |
| 12 | 13.903 | 1359.22 | 88.11 |
| 13 | 14.113 | 1046.2 | 84.5 |
| 14 | 15.114 | 9445.97 | 105.98 |
| 15 | 16.206 | 3847.13 | 114.97 |
| 16 | 16.736 | 4171.49 | 77.61 |
| 17 | 18.619 | 7938.15 | 112.43 |
| 18 | 22.137 | 13159.29 | 139.24 |
| 19 | 24.676 | 3591.43 | 35.14 |
| 20 | 26.902 | 7333.99 | 27.14 |

The hepatocellular carcinoma cell line used in the example is HepG2, and the manipulations of the cell culture and treatment are as described in Example 2. The cells were grouped into a control group (without treatment) and the experiment groups treated with 1× leaf juice, 0%, 0.001%, 0.01%, 0.1%, 0.5% or 1% of Fraction #9 as mentioned in Example 8. Each group was repeated four to eight times.

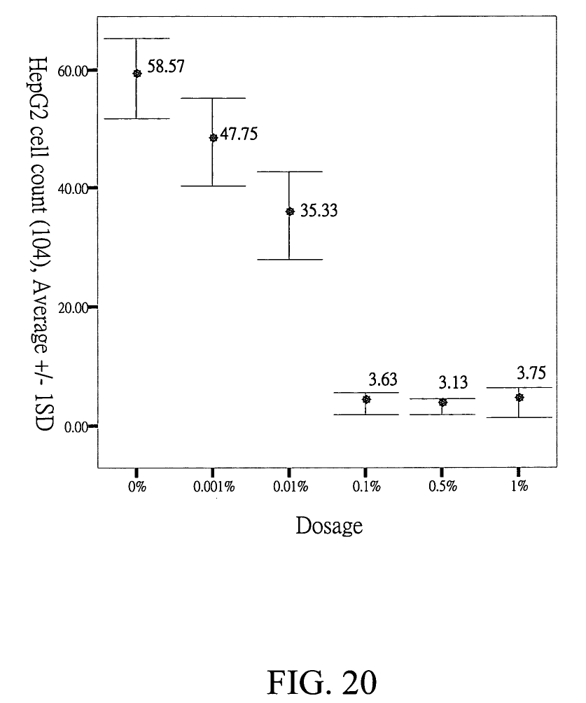

Cell count of HepG2 cell of each group on day 4 was determined and shown in FIG. 20. Referring to FIG. 20, 1% (P<0.001), 0.5% (P<0.001), 0.1% (P<0.001) and 0.01% (P<0.001) of Fraction #9 show significant inhibiting effect. Additionally, 0.001% (P=0.092) of Fraction #9 inhibits cell growth nearly significantly. The inhibiting rates of 1%, 0.5%, 0.1%, 0.01%, and 0.001% of Fraction #9 are 93.6%, 94.7%, 93.8%, 39.7%, and 18.5%, respectively. The cell number inhibited per percentage of Fraction #9 is 43.65×10⁴.

### EXAMPLE 10

### Effects of Fractions of Leaf Juice in Inhibiting Huh7 Cell Growth

The fractions of leaf juice are as prepared in Example 8.

Treatment: The cell line used for screening the fractions of leaf juice is and Huh7. The manipulation of the cell lines is as described in Example 2 and the cell numbers were counted on day 4.

The cell viability of Huh7 on day 4 is illustrated in Table 7.

TABLE 7

| | 1% of drug | 5% of drug |
|---|---|---|
| Control | 100 | 100 |
| 1 X leaf juice | 100 | 39 |
| 25 X leaf juice | 33 | 0 |
| Fraction #1 | 83 | 39 |
| Fraction #2 | 0 | 6 |
| Fraction #9 | 13 | 0 |

It shows that Fractions #1, #2 and #9 inhibit tumor growth with dose dependency.

1× and 25× leaf juice, 0%, 0.01%, 0.1%, 0.5%, 1% and 3% of Fractions #1, #2 and #9 were further used for treating Huh7 cells. Each group was repeated four to seven times.

The result shows that 1% (P<0.01) and 3% (P=0.001) Fraction #1, 1% (P<0.05) and 3% (P<0.05) Fraction #2, 0.01% Fraction #9 (P=0.01), and 0.5% (P<0.05), 1% (P<0.05) and 3% (P<0.01) 25× leaf juice inhibit Huh7 growth significantly. On the other hand, 1% (P=0.937) and 3% (P=0.666) 1× leaf juice do not show significant inhibiting effect. It evidenced that Fraction #9 has the strongest effect where the effective dosage is only 0.01%. 25× leaf juice also has strong effect where the effective dosage is 0.5%. The effective dosages of Fractions #1 and #2 are 1%. The inhibiting rates of 1% and 3% Fraction #1, 1% and 3% Fraction #2, 0.01 Fraction #9, and 0.5%, 1% and 3% 25× leaf juice are 59.3%, 76.2%, 53.9%, 64.2%, 53.7%, 49.9%, 53.9%, and 67.1% respectively.

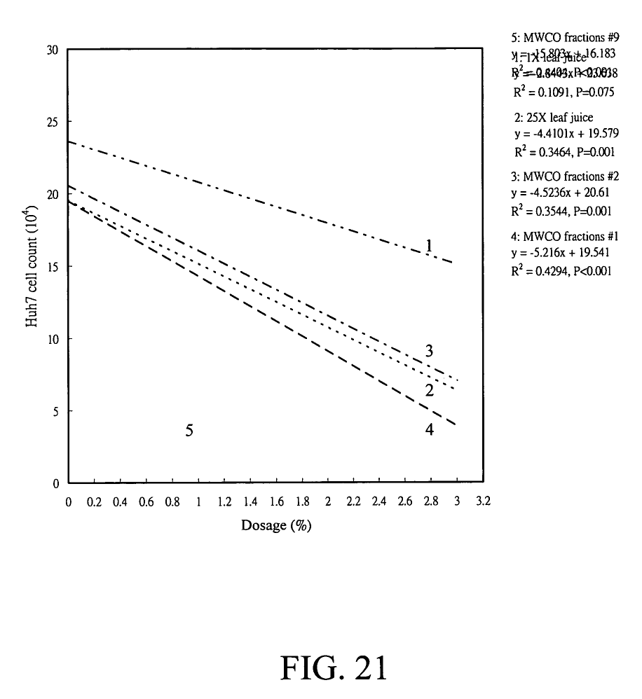

The cell numbers inhibited per percentage of 1× and 25× leaf juice and Fractions #1, #2 and #9 are shown in FIG. 21.

It shows that 1× (P=0.001, R²=0.346) leaf juice and Fractions #1 (P<0.001, R²=0.429), #2 (P=0.001, R²=0.354) and #9 (P<0.001, R²=0.440) significantly inhibit HepG2 growth with dose dependency. The cell numbers inhibited per percentage of 25× leaf juice and Fractions #1, #2 and #9 on day 4 are 4.41, 5.22, 4.52, 15.8×10⁴, respectively. The inhibiting effects are Fraction #9 >Fraction #1 >Fraction #2>25× leaf juice.

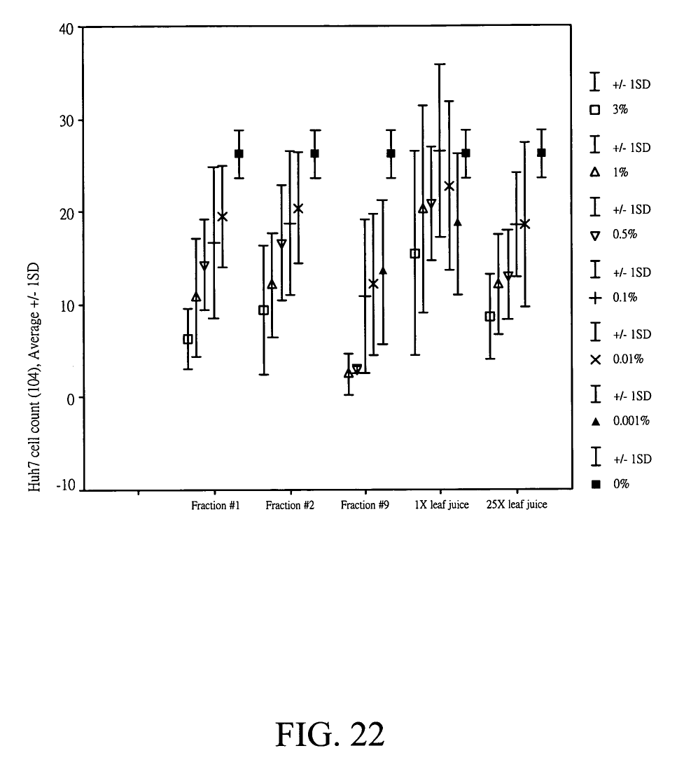

The effects of 1× and 25× leaf juice, Fractions #1, #2 and #9 with the same dosage are also analyzed. The result is shown in FIG. 22.

When the dosages are 1% (P<0.01) and 0.5% (P<0.01), the effect of Fraction #9 is stronger than that of 1× leaf juice significantly. Furthermore, when the dosage is 0.5%, the effect of Fraction #9 is stronger than that of Fraction #2 significantly (P=0.05).

### EXAMPLE 11

### Fraction #9 of Leaf Juice for Treating Hepatocellular Carcinoma Huh7

The hepatocellular carcinoma cell line used in the example is Huh7, and the manipulations of the cell culture and treatment are as described in Example 2. The cells were grouped into a control group (without treatment) and experiment groups treated with 1× leaf juice, 0%, 0.001%, 0.01%, 0.1%, 0.5% or 1% of Fraction #9 as mentioned in Example 10. Each group was repeated four to six times.

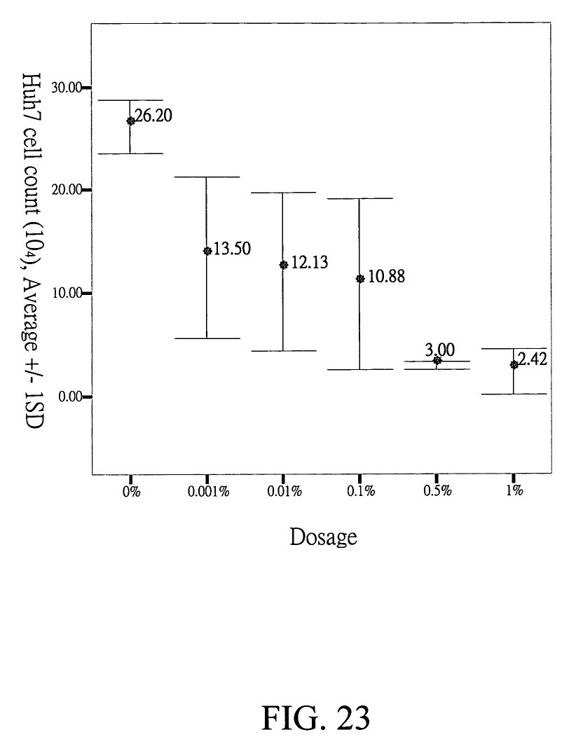

Cell count of Huh7 cells of each group on day 4 was determined and shown in FIG. 23. Referring to FIG. 23, 1% (P<0.001), 0.5% (P<0.001), 0.1% (P<0.05) and 0.01% (P<0.05) of Fraction #9 show significant inhibiting effect. Additionally, 0.001% (p =0.067) of Fraction #9 inhibits cell growth nearly significantly. The inhibiting rates of 1%, 0.5%, 0.1%, 0.01%, and 0.001% of Fraction #9 are 90.8%, 88.6%, 58.5%, 53.7%, and 48.5%, respectively. The cell number inhibited per percentage of Fraction #9 is 15.8×10⁴.

While embodiments of the present invention have been illustrated and described, various modifications and improvements can be made by persons skilled in the art. The embodiments of the present invention are therefore described in an illustrative but not restrictive sense. It is intended that the present invention is not limited to the particular formns as illustrated, and that all the modifications not departing from the spirit and scope of the present invention are within the scope as defined in the appended claims.

What is claimed is:

1. A composition for treating cancer and/or tumor comprising an effective amount of the leaf juice of Plectranthus amboinicus.

2. The composition of claim 1, for treating malignant tumor.

3. The composition of claim 1, wherein the tumor is a p53 related tumor.

4. The composition of claim 1, wherein the tumor comprises hepatocellular carcinoma and melanoma.

5. The composition of claim 1, wherein a spectrogram of 50 μL of the leaf juice of Plectranthus amboinicus taken at 214 nm through high performance liquid chromatography (HPLC) using a ZORBAX™ C18 column with an inner diameter (I.D.) of 4.6 mm, a length (L) of 15 cm comprises peaks at retention time of 1.756, 2.573, 7.118, 7.851, 9.715, 10.278, 10.864, 11.212, 12.287, 12.799, 13.178, 13.413, 14.027, 14.794, 16.253, and 18.742 minutes, where the leaf juice is separated with a linear gradient elution of 0-30% acetonitrile and 0.1% TFA in 30 min and a flow rate is 1 mL/min.

6. The composition of claim 1, which comprises an effective amount of the fraction of the leaf juice of Plectranthus amboinicus of a molecular weight more than 50 kD.

7. The composition of claim 1, further comprising an antineoplastic agent.

8. The composition of claim 7, wherein the antineoplastic agent is paciltaxel.

9. The composition of claim 1, which is formulated in a pharmaceutically acceptable carrier.

10. The composition of claim 1, which is applied to a subject via injection, oral administration, or topical administration.

11. A method for producing the composition of claim 1, wherein the leaf juice is produced by a method comprising the steps of:
(a) harvesting Plectranthus amboinicus leaves;
(b) washing the leaves in step (a) with distilled water;
(c) removing the water from the leaves; and
(d) obtaining the leaf juice from the leaves in step (c).

12. The method of claim 11, wherein the method for producing the leaf juice further comprises step (d2) of centrifuging the leaf juice in step (d) to remove the tissue fiber.

13. The method of claim 11, wherein the method for producing the leaf juice further comprises a step of sterilizing the leaf juice.

14. The method of claim 13, wherein the leaf juice is sterilized by filtration.

15. The method of claim 11, wherein the method for producing the leaf juice further comprises a step of concentrating the leaf juice.

16. A composition for treating cancer and/or tumor fraction comprising an effective amount of the fraction of the leaf juice of Plectranthus amboinicus of a molecular weight more than 50 kD.

17. The composition of claim 16, wherein a spectrogram of 50 μL of the fraction of a molecular weight more than 50 kD taken at 214 nm through high performance liquid chromatography (HPLC) using a ZORBAX™ C18 column with an inner diameter (I.D.) of 4.6 mm, a length (L) of 15 cm comprises peaks at retention time of 2.574, 4.798, 5.728, 7.101, 9.701, 10.279, 10.808, 11.189, 12.235, 13.35, 13.584, 13.903, 14.113, 15.114, 16.206, 16.736, 18.619, 22.137, 24.676, and 26.902 minutes, where the fraction of a molecular weight more than 50 kD is separated with a linear gradient elution of 0-30% acetonitrile and 0.1% TFA in 30 min and a flow rate is 1 mL/min.

18. The composition of claim 16, for treating malignant tumor.

19. The composition of claim 16, wherein the tumor is a p53 related tumor.

20. The composition of claim 16, wherein the tumor comprises hepatocellular carcinoma and melanoma.

21. The composition of claim 16, further comprising an antineoplastic agent.

22. The composition of claim 21, wherein the antineoplastic agent is paciltaxel.

23. The composition of claim 16, which is formulated in a pharmaceutically acceptable carrier.

24. The composition of claim 16, which is applied to a subject via injection, oral administration, or topical administration.

25. A method for producing the composition of claim 16, wherein the fraction of the leaf juice of a molecular weight more than 50 kD is produced by a method comprising the steps of:
(a) harvesting Plectranthus amboinicus leaves;
(b) washing the leaves in step (a) with distilled water;
(c) removing water from the leaves;
(d) obtaining the leaf juice from the leaves in step (c); and
(e) passing the leaf juice in step (d) through a filter for removing the fraction of the leaf juice of a molecular weight less than 50 kD and obtaining the fraction of a molecular weight more than 50 kD.

26. The method of claim 25, wherein the method for producing the fraction of the leaf juice of a molecular weight more than 50 kD further comprises step (d2) of centrifuging the leaf juice in step (d) to remove the tissue fiber.

27. The method of claim 25, wherein the method for producing the fraction of the leaf juice of a molecular weight more than 50 kD further comprises a step of sterilizing the molecular weight more than 50 kD fraction of the leaf juice.

28. The method of claim 25, wherein the fraction of the leaf juice of a molecular weight more than 50 kD is sterilized by filtration.

29. The method of claim 25, wherein the method for producing the fraction of the leaf juice of a molecular weight more than 50 kD further comprises a step of concentrating the molecular weight more than 50 kD fraction the leaf juice.

30. A method for treating cancer and/or tumor comprising administering the composition of claim 1 to a subject in need of such therapy.

31. A method for treating cancer and/or tumor comprising administering the composition of claim 16 to a subject in need of such therapy.
